(19) United States
(12) Patent Application Publication
(10) Pub. No.: US 2004/0235728 A1
(43) Pub. Date: Nov. 25, 2004

(54) Compositions and methods for treating osteoporosis

(76) Inventors: Selwyn Aubrey Stoch, Clark, NJ (US); John Orloff, Princeton Junction, NJ (US)

Correspondence Address: MERCK AND CO INC, P O BOX 2000, RAHWAY, NJ, 070650907

(21) Appl. No.: 10/494,542

(22) PCT Filed: Nov. 4, 2002
(86) PCT No.: PCT/US02/35341

Related U.S. Application Data
(60) Provisional application No. 60/337,785, filed on Nov. 8, 2001.

(51) Int. Cl. A61K 38/28; A61K 31/66; A61K 39/395; A61K 31/60; A61K 31/415

(52) U.S. Cl. 514/12; 514/102; 514/165; 514/406; 424/145.1

(57) ABSTRACT

The present invention relates to pharmaceutical compositions comprising a cathepsin K inhibitor which are useful for treating such conditions as bone resorption, osteoporosis, arthritis, tumor metastases, Paget's disease, and other metabolic bone disorders characterized by increased bone resorption.

### BACKGROUND OF THE INVENTION

A variety of disorders in humans and other mammals involve or are associated with abnormal bone resorption. Such disorders include, but are not limited to, osteoporosis, Paget's disease, periprosthetic bone loss or osteolysis, and hypercalcemia of malignancy. The most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 70% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

Osteoporosis is characterized by progressive loss of bone architecture and mineralization leading to the loss in bone strength and an increased fracture rate. The skeleton is constantly being remodeled by a balance between osteoblasts that lay down new bone and osteoclasts that breakdown, or resorb, bone. In some disease conditions and advancing age the balance between bone formation and resorption is disrupted; bone is removed at a faster rate. Such a prolonged imbalance of resorption over formation leads to weaker bone structure and a higher risk of fractures.

Bone resorption is primarily performed by osteoclasts, which are multinuclear giant cells. Osteoclasts resorb bone by forming an initial cellular attachment to bone tissue, followed by the formation of an extracellular compartment or lacunae. The lacunae are maintained at a low pH by a proton-ATP pump. The acidified environment in the lacunae allows for initial demineralization of bone followed by the degradation of bone proteins or collagen by proteases such as cysteine proteases. See Delaisse, J. M. et al., 1980, Biochem J 192:365-368; Delaisse, J. et al., 1984, Biochem Biophys Res Commun:441-447; Delaisse, J. M. et al., 1987, Bone 8:305-313, which are hereby incorporated by reference in their entirety. Collagen constitutes 95% of the organic matrix of bone. Therefore, proteases involved in collagen degradation are an essential component of bone turnover, and as a consequence, the development and progression of osteoporosis.

Cathepsins belong to the papain superfamily of cysteine proteases. These proteases function in the normal physiological as well as pathological degradation of connective tissue. Cathepsins play a major role in intracellular protein degradation and turnover and remodeling. To date, a number of cathepsin have been identified and sequenced from a number of sources. These cathepsins are naturally found in a wide variety of tissues. For example, cathepsin B, F, H, L, K, S, W, and Z have been cloned. Cathepsin K (which is also known by the abbreviation cat K) is also known as cathepsin O and cathepsin O2. See PCT Application WO 96/13523, Khepri Pharmaceuticals, Inc., published May 9, 1996, which is hereby incorporated by reference in its entirety. Cathepsin L is implicated in normal lysosomal proteolysis as well as several disease states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immunbe responses, including, but not limited to, rejection of organ transplants or tissue grafts. Increased Cathepsin B levels and redistribution of the enzyme are found in tumors, suggesting a role in tumor invasion and matastasis. In addition, aberrant Cathpsin B activity is implicated in such disease states as rheumatoid arthritis, osteoarthritis, pneumocystisis carinii, acute pancreatitis, inflammatory airway disease and bone and joint disorders.

Cysteine protease inhibitors such as E-64 (trans-epoxysuccinyl-L-leucylamide-(4-guanidino) butane) are known to be effective in inhibiting bone resorption. See Delaisse, J. M. et al., 1987, Bone 8:305-313, which is hereby incorporated by reference in its entirety. Recently, cathepsin K was cloned and found specifically expressed in osteoclasts See Tezuka, K. et al., 1994, J Biol Chem 269:1106-1109; Shi, G. P. et al., 1995, FEBS Lett 357:129-134; Bromme, D. and Okamoto, K., 1995, Biol Chem Hoppe Seyler 376:379-384; Bromme, D. et al., 1996, J Biol Chem 271:2126-2132; Drake, F. H. et al., 1996, J Biol Chem 271:12511-12516, which are hereby incorporated by reference in their entirety. Concurrent to the cloning, the autosomal recessive disorder, pycnodysostosis, characterized by an osteopetrotic phenotype with a decrease in bone resorption, was mapped to mutations present in the cathepsin K gene. To date, all mutations identified in the cathepsin K gene are known to result in inactive protein. See Gelb, B. D. et al., 1996, Science 273:1236-1238; Johnson, M. R. et al., 1996, Genome Res 6:1050-1055, which are hereby incorporated by reference in their entirety. Therefore, it appears that cathepsin K is involved in osteoclast mediated bone resorption.

Cathepsin K is synthesized as a 37 kDa pre-pro enzyme, which is localized to the lysosomal compartment and where it is presumably autoactivated to the mature 27 kDa enzyme at low pH. See McQueney, M. S. et al., 1997, J Biol Chem 272:13955-13960; Littlewood-Evans, A. et al., 1997, Bone 20:81-86, which are hereby incorporated by reference in their entirety. Cathepsin K is most closely related to cathepsin S having 56% sequence identity at the amino acid level. The S₂P₂ substrate specificity of cathepsin K is similar to that of cathepsin S with a preference in the P1 and P2 positions for a positively charged residue such as arginine, and a hydrophobic residue such as phenylalanine or leucine, respectively. See Bromme, D. et al., 1996, J Biol Chem 271: 2126-2132; Bossard, M. J. et al., 1996,J Biol Chem 271:12517-12524, which are hereby incorporated by reference in their entirety. Cathepsin K is active at a broad pH range with significant activity between pH 4-8, thus allowing for good catalytic activity in the resorption lacunae of osteoclasts where the pH is about 4-5.

Human type I collagen, the major collagen in bone is a good substrate for cathepsin K. See Kafienah, W., et al., 1998, Biochem J 331:727-732, which is hereby incorporated by reference in its entirety. In vitro experiments using antisense oligonucleotides to cathepsin K, have shown diminished bone resorption in vitro, which is probably due to a reduction in translation of cathepsin K mRNA. See Inui, T., et al., 1997, J Biol Chem 272:8109-8112, which is hereby incorporated by reference in its entirety. The crystal structure of cathepsin K has been resolved. See McGrath, M. E., et al., 1997, Nat Struct Biol 4:105-109; Zhao, B., et al., 1997, Nat Struct Biol 4: 109-11, which are hereby incorporated by reference in their entirety. Also, selective peptide based inhibitors of cathepsin K have been developed See Bromme, D., et al., 1996, Biochem J 315:85-89; Thompson, S. K., et al., 1997, Proc Natl Acad Sci USA 94:14249-14254, which are hereby incorporated by reference in their entirety. Accordingly, inhibitors of Cathepsin K can reduce bone resorption. Such inhibitors would be useful in treating disorders involving bone resorption, such as osteoporosis.

Combination therapy has a number of benefits including more effectively treating the underlying bone loss of conditions such as osteoporosis, Paget's disease, periprosthetic bone loss or osteolysis, and hypercalcemia of malignancy. Combination therapy can also prove beneficial in treating or preventing arthritic condition, especially in treating or preventing osteoarthritis and rheumatoid arthritis, including the prevention of subchondral bone loss, osteophyte formation, and ultimately joint deterioration/destruction, and in the prevention and treatment of metatstatic bone disease. The advantage of combination therapy can include a benefit that is not seen with monotherapy.

### SUMMARY OF THE INVENTION

The present invention relates to a pharmaceutical composition comprising a cathepsin K inhibitor or a pharmacaeutically acceptable salt thereof and and one or more active ingredients selected from the following:

a) an organic bisphosphonate or a pharmaceutically acceptable salt or ester thereof,

b) an estrogen receptor modulator,

c) an androgen receptor modulator,

d) a steroid with mixed estrogenic-progestogenic-androgenic properties,

e) a cytotoxic antiproliferative agent,

f) a matrix metalloproteinase inhibitor,

g) an inhibitor of epidermal-derived growth factors, an inhibitor of fibroblast-derived growth factors, an inhibitor of platelet-derived growth factors,

h) an inhibitor of VEGF,

i) an antibody to a growth factor, an antibody to a growth factor receptor,

j) an inhibitor of Flk-1/KDR, an inhibitor of Flt-1, an inhibitor of Tck/Tie-2, an inhibitor of Tie-1,

k) αvβ3 receptor antagonist,

l) growth hormone, a growth hormone analogue, a growth hormone secretagogue,

m) an inhibitor of osteoclast ATPase,

n) an inhibitor of urokinase plasminogen activator,

o) a tumor-specific antibody-interleukin-2 fusion protein,

p) an inhibitor of HMG-CoA reductase,

q) an inhibitor of p38 kinase,

r) an activator of the peroxisome proliferator-activated receptor-γ,

s) a prenylation inhibitor,

t) a COX-1 inhibitor, COX-2 inhibitor, a dual COX-1/COX-2 inhibitor,

u) a calcilytic,

v) growth factors

w) Parathyroid hormone (PTH), PTH fragments, PTH analogues,

x) Parathyroid hormone-related protein (PTHrP), PTHrP fragments, PTHrP analogues,

y) a prostanoid EP2 receptor agonist, a non-prostanoid EP2 receptor agonist, and mixtures thereof,

z) inducers of osteoblastic Cbfa-1,

aa) a bone anabolic agent that directly stimulates osteoblastic activity,

bb) an inhibitor of tumor necrosis factor-α, and

cc) an anti-inflammatory agent,

dd) a Dkk inhibitor or a stimulator of the Wnt signaling pathway,

ee) a Y2 receptor antagonist,

ff) a central inhibitor of leptin signaling,

gg) a sclerostin antagonist,

hh) a P2X7 receptor agonist,

ii) a CIC-inhibitor,

and the pharmaceutically acceptable salts and mixtures thereof.

In further embodiments, the present invention relates to a method for inhibiting bone resorption in a mammal in need thereof comprising administering one of the compositions of the present invention.

In further embodiments, the present invention relates to a method for inhibiting bone resorption in a mammal in need thereof comprising sequentially administering the components of the pharmaceutical compositions of the present invention.

In further embodiments, the present invention relates to a method for inhibiting bone resorption in a mammal in need thereof comprising concomitantly administering the components of the pharmaceutical compositions of the present invention.

In further embodiments, the present invention relates to the use of a composition of the present invention in the manufacture of a medicament for inhibiting bone resorption in a mammal in need thereof.

In further embodiments, the present invention relates to the use of a composition of the present invention for inhibiting bone resorption in a mammal in need thereof.

All percentages and ratios used herein, unless otherwise indicated, are by weight. The invention hereof can comprise, consist of, or consist essentially of the essential as well as optional ingredients, components, and methods described herein.

### DETAILED DESCRIPTION OF THE INVENTION

The present invention relates to compositions and methods for inhibiting bone resorption in a mammal in need of such treatment.

The term “pharmaceutically effective amount”, as used herein, means that amount of a compound or composition, that will elicit the desired therapeutic effect or response or provide the desired benefit when administered in accordance with the desired treatment regimen. A preferred pharmaceutically effective amount is a bone resorption inhibiting amount.

The present invention relates to inhibiting bone resorption, or more specifically to inhibiting undesired or abnormal bone resorption. The term “abnormal bone resorption”, as used herein means a degree of bone resorption that exceeds the degree of bone formation, either locally, or in the skeleton as a whole. Alternatively, “abnormal bone resorption” can be associated with the formation of bone having an abnormal structure, as in Paget's disease.

The term “bone resorption inhibiting”, as used herein, means preventing bone resorption by the direct or indirect alteration of osteoclast formation or activity. Inhibition of bone resorption refers to prevention of bone loss, especially the inhibition of removal of existing bone either from the mineral phase and/or the organic matrix phase, through direct or indirect alteration of osteoclast formation or activity.

The term “until the desired therapeutic effect is achieved”, as used herein, means that the therapeutic agent or agents are continuously administered, according to the dosing schedule chosen, up to the time that the clinical or medical effect sought for the disease or condition being treated is observed by the clinician or researcher. For methods of treatment of the present invention, the pharmaceutical composition is continuously administered until the desired change in bone mass or structure is observed. In such instances, achieving an increase in bone mass or a replacement of abnormal bone structure with normal bone structure are the desired objectives. For methods of prevention of the present invention, the pharmaceutical composition is continuously administered for as long as necessary to prevent the undesired condition. In such instances, maintenance of bone mass density is often the objective. Nonlimiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

The term “Cathepsin K inhibitor”, as used herein, refers to a compound which is a potent and selective inhibitor of cathepsin K, a lysosomal cysteine protease that is highly expressed in osteoclasts and implicated in bone resorption.

The term “bone resorption,” as used herein, refers to the process by which osteoclasts degrade bone.

Illustrating the invention is a pharmaceutical composition comprising components of the present invention and a pharmaceutically acceptable carrier. Another illustration of the invention is a process for making a pharmaceutical composition comprising combining two or more components described above and a pharmaceutically acceptable carrier.

Further illustrating the invention is a method of treating and/or preventing a condition mediated by inhibiting the cathepsin K enzyme in a mammal in need thereof, comprising administering to the mammal a therapeutically effective amount of the compositions described above.

The pharmaceutical compositions of the present invention are useful for treating such conditions as bone resorption, osteoporosis, arthritic conditions, osteoarthritis, rheumatoid arthritis, tumor metastases, breast cancer, prostate cancer, metastatic bone disease, Paget's disease, and other metabolic bone disorders characterized by increased bone resorption.

Exemplifying the invention are compositions comprising a cathpesin K inhibitor and one or more active ingredients selected from the following:

a) an organic bisphosphonate or a pharmaceutically acceptable salt or ester thereof,

b) an estrogen receptor modulator,

c) an androgen receptor modulator,

d) a steroid with mixed estrogenic-progestogenic-androgenic properties,

e) a cytotoxic antiproliferative agent,

f) a matrix metalloproteinase inhibitor,

g) an inhibitor of epidermal-derived growth factors,

h) an inhibitor of fibroblast-derived growth factors,

i) an inhibitor of platelet-derived growth factors,

j) an inhibitor of VEGF,

k) an antibody to a growth factor, an antibody to a growth factor receptor,

l) an inhibitor of Flk-1/KDR,

m) an inhibitor of Flt-1,

n) an inhibitor of Tck/Tie-2,

o) an inhibitor of Tie-1,

p) αvβ3 receptor antagonist,

q) growth hormone,

r) a growth hormone analogue,

s) a growth hormone secretagogue,

t) an inhibitor of osteoclast ATPase,

u) an inhibitor of urokinase plasminogen activator,

v) a tumor-specific antibody-interleukin-2 fusion protein,

w) an inhibitor of HMG-CoA reductase,

x) an inhibitor of p38 kinase,

y) an activator of the peroxisome proliferator-activated receptor-y,

z) a prenylation inhibitor,

aa) a COX-1 inhibitor,

bb) COX-2 inhibitor,

cc) a dual COX-1/COX-2 inhibitor,

dd) a calcilytic,

ee) growth factors

ff) Parathyroid hormone (PTH),

gg) PTH fragments,

hh) PTH analogues,

ii) Parathyroid hormone-related protein (PTHrP),

jj) PTHrP fragments,

kk) PTHrP analogues,

ll) a prostanoid EP2 receptor agonist,

mm) a non-prostanoid EP2 receptor agonist,

nn) inducers of osteoblastic Cbfa-1,

oo) a bone anabolic agent that directly stimulates osteoblastic activity,

pp) an inhibitor of tumor necrosis factor-α, and

qq) an anti-inflammatory agent,

rr) a Dkk inhibitor or a stimulator of the Wnt signaling pathway,

ss) a Y2 receptor antagonist,

tt) a central inhibitor of leptin signaling,

uu) a sclerostin antagonist,

vv) a P2X7 receptor agonist,

ww) a CIC-7 inhibitor,

and the pharmaceutically acceptable salts and mixtures thereof.

Further exemplifying the invention, the active ingredient is selected from a cathepsin K inhibitor and one more active ingredients selected from the following:

a) an organic bisphosphonate or a pharmaceutically acceptable salt or ester thereof,

b) an estrogen receptor modulator,

c) an androgen receptor modulator,

d) an inhibitor of osteoclast proton ATPase,

e) an inhibitor of HMG-CoA reductase,

f) an αᵥβ₃ receptor antagonist; and mixtures thereof, and

g) an osteoblast anabolic agent, such as PTH;

and the pharmaceutically acceptable salts and mixtures thereof.

Nonlimiting examples of cathepsin K inhibitors can be found in PCT publications WO 00/55126 to Axys Pharmaceuticals and WO 01/49288 to Merck Frosst Canada & Co. and Axys Pharmaceuticals.

Nonlimiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

Nonlimiting examples of estrogen receptor modulators include estrogen, progestogen, estradiol, droloxifene, raloxifene, lasofoxifene, TSE-424 and tamoxifen. Nonlimiting examples of estrogen receptor modulators can also be found in International Publication Nos. WO 01/82923 (published Nov. 8, 2001) and WO 02/41835 (published May 30, 2002), assigned to Merck & Co., Inc. which are hereby incorporated by reference in their entirety.

Nonlimiting examples of cytotoxic/antiproliferative agents are taxol, vincristine, vinblastine, cyclophosphamide, and doxorubicin.

Nonlimiting examples of anti-inflammatory agents include traditional NSAIDs, rofecoxib, celecoxib, azathioprine, penicillamine, methotrexate, sulfasalazine, prednisone, leflunomide, infliximab, and etanercept.

Nonlimiting examples of integrin receptor antagonists, and methods for their preparation, are found in U.S. Pat. No. 5,925,655 (issued Jul. 20, 1999), U.S. Pat. No. 6,211,184 (issued Apr. 3, 2001), U.S. Pat. No. 5,919,792 (issued Jul. 6, 1999), U.S. Pat. No. 5,952,792 (issued Sep. 14, 1999), U.S. Pat. No. 6,017,925 (issued Jan. 25, 2000), U.S. Pat. No. 6,048,861 (issued Apr. 11, 2000), U.S. Pat. No. 6,232,308 (issued May 15, 2001), U.S. Pat. No. 6,358,970 (issued Mar. 19, 2002), U.S. Pat. No. 6,040,311 (issued Mar. 21, 2000), U.S. Pat. No. 6,066,648 (issued May 23, 2000), U.S. Pat. No. 6,211,191 (issued Apr. 3, 2001), U.S. Pat. No. 6,017,926 (issued Jan. 25, 2000), U.S. Pat. No. 6,090,944 (Jul. 18, 2000), U.S. Pat. No. 6,410,526 (issued Jun. 25, 2002), U.S. Pat. No. 6,413,955 (issued Jul. 2, 2002), U.S. Pat. No. 6,426,353 (issued Jul. 30, 2002), U.S. Pat. No. 6,444,680 (issued Sep. 3, 2002), and in PCT International Publication Numbers WO 00/48603 (published Aug. 24, 2000), WO 01/53297 (published Jul. 26, 2001), WO 01/53262 (published Jul. 26, 2001), WO 02/22616 (published Mar. 21, 2002), WO 02/07730 (published Jan. 31, 2002), WO 02/28840 (published Apr. 11, 2002), WO 02/40505 (published May 23, 2002).

Evidence has been presented that androgenic steroids play a physiological role in the development of bone mass in men and women and that androgens act directly on bone. Androgen receptors have been demonstrated in human osteoblast-like cell lines and androgens have been shown to directly stimulate bone cell proliferation and differentiation. For a discussion, reference is made to S.R. Davis, “The therapeutic use of androgens in women,” J. Steroid Biochem. Mol. Biol., 69: 177-184 (1999) and K. A. Hansen and S.P.T. Tho, “Androgens and Bone Health,” Seminars in Reproductive Endocrinology,” 16: 129-134 (1998). Thus, androgen receptor modulators may have utility in the treatment and prevention of bone loss in women.

Data exists on the use of synthetic steroids with mixed estrogenic-progestogenic-androgenic properties that affect bone. Tibolone is one such example, that has been shown in clinical trials to prevent bone loss in the spine and proximal hip in early- and late-postmenopausal women. For a discussion, reference is made to B Berning et al., “Tibolone and its effects on bone: a review,” Climacteric, 4: 120-136 (2001). Flavanoids (ipriflavone) have also been shown to partially inhibit bone loss [see M. Gambacciani et al., “Effects of combined low dose of the isoflavone derivative ipriflavone and estrogen replacement on bone mineral density and metabolism in postmenopausal women,” Maturitas 28: 75-81 (1997) and C. Gennari et al., “Effect of chronic treatment with ipriflavone in postmenopausal women with low bone mass,” Calcif Tiss Int 61: S19-S22 (1997)].

The activation and induction of matrix metalloproteinases (MMPs) appear to be involved in bone resorption and the accompanying degradation of bone matrix. MMPs also play a role in arthritis, including rheumatoid arthritis (RA), an autoimmune disorder characterized by inflammation and destruction of cartilage and bone. Evidence indicates that the joint destruction in RA is mediated by cytokines, matrix-degrading enzymes and other molecules (See H. B. Sun and H. Yokota, “Messenger-RNA expression of matrix metalloproteinases, tissue inhibitors of metalloproteinases, and transcription factors in rheumatic synovial cells under mechanical stimuli,” Bone 28: 303-309 (2000)). The identification of aggrecanase-1, a metalloproteinase, may also serve as a potential target for drug development to prevent the loss of cartilage in arthritis (see M. D. Tortorealla et al, “Purification and cloning of aggrecenase-1: a member of the ADAMTS family of proteins,” Science 284: 1664-1666 (1999)). Matrix metalloproteinases, particularly MMP-2, represent a target for the development of antitumor therapy to prevent tumor invasion and the formation of metastases (see E. Morgunova et al., “Structure of human pro-matrix metalloproteinase-2: activation mechanism revealed,” Science 284: 1667-1670 (1999). Inhibitors of MMP-2 binding to the αᵥβ₃ integrin may provide promise in treating diseases of uncontrolled angiogenesis (see S. Stilletti et al., “Disruption of the matrix metalloproteinase 2 binding to integrin αᵥβ₃ by an organic molecule inhibits angiogenesis and tumor growth in vivo,” PNAS 98: 119-124 (2001). Thus inhibitors of matrix metalloproteinases may hold utility in treating arthritis, bone resorption and metastatic bone disease.

Basic fibroblast growth factor (FGF-2) is an important modulator of bone cell function and it has been reported to restore bone mass in ovariectomized rats [see H. Liang et al., “Bone anabolic effects of basic fibroblast growth factor in ovariectomized rats,” J Clin End Metab 140: 5780-5788 (1999)]. Furthermore, disruption FGF-2 results in decreased bone formation and decreased bone mass (see A. Montero et al., “Disruption of the fibroblast growth factor-2 gene results in decreased bone mass and bone formation,” J Clin Invest 105: 1085-1093 (2000). It has also been shown that local and systemic administration of acidic fibroblast growth factor (FGF-1) restores bone microarchitecture and prevents bone loss associated with estrogen withdrawal [see C. R. Dunstan et al., “Systemic administration of acidic fibroblast growth factor (FGF-1) prevents bone loss and increases new bone formation in ovariectomized rats,” J Bone Miner Res 14: 953-959 (1999)].

Platelet derived growth factor (PDGF) has mitogenic activities and its release may play a role in fracture repair [see E. Canalis and S. Rydziel, “Platelet-derived growth factor and the skeleton,” In: Principles of Bone Biology Ed: Bilezikian, Raisz and Rodan, Academic Press (1996)]. Growth factors, including PDGF, epidermal growth factor (EGF) and IGF-1 have been shown to increase endochondral ossification in neonatal rat metatarsals [see G. Krishnan et al., “Effect of growth factors such as IGF-1, EGF, PDGF-BB, and FGF-2 on the neonatal rat metatarsal organ culture model,” J Bone Miner Res S264 (2000)].

The angiogenic factor VEGF has been shown to stimulate the bone-resorbing activity of isolated mature rabbit osteoclasts via binding to its receptors on osteoclasts (see M. Nakagawa et al., “Vascular endothelial growth factor (VEGF) directly enhances osteoclastic bone resorption and survival of mature osteoclasts,” FEBS Letters, 473: 161-164 (2000)). Therefore, the development of antagonists of VEGF binding to osteoclast receptors, such as KDR/Flk-1 and Flt-1, may provide yet a further approach to the treatment or prevention of bone resorption.

αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact non-covalently and bind extracellular matrix ligands in a divalent cation-dependent manner. The most abundant integrin on osteoclasts is αᵥβ₃ (>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

Growth hormone (GH) increases bone turnover and stimulates osteoblast activity and has been shown to increase the strength of cortical bone in aged rats [see TT Andreassen et al., “Growth hormone stimulates bone formation and strength of cortical bone in aged rats,” J Bone Miner Res 10: 1057-1067 (1995)]. Continued use of recombinant human growth hormone (rhGH) increases BMD by 18 months in growth hormone deficient adults [see G. Johannson et al., “Two years of growth hormone (GH) increases bone mineral content and density in hypopituitary patients with adult-onset GH deficiency J Clin End Metab 81: 2865-2873]. Furthermore, orally active GH secretagogues have been shown to increase femoral neck BMD [see M. G. Murphy et al., “Effect of alendronate and MK-677 (a growth hormone secretagogue), individually and in combination, on markers of bone turnover and bone mineral density in postmenopausal osteoporotic women,” J Clin Endo Metab 86: 1116-1125 (2001)].

The proton ATPase which is found on the apical membrane of the osteoclast has been reported to play a significant role in the bone resorption process. Therefore, this proton pump represents an attractive target for the design of inhibitors of bone resorption which are potentially useful for the treatment and prevention of osteoporosis and related metabolic diseases (see C. Farina et al., “Selective inhibitors of the osteoclast vacuolar proton ATPase as novel bone antiresorptive agents,” DDT, 4: 163-172 (1999)).

Evidence for crucial role of the urokinase-urokinase receptor (u-PA-u-PAR) in angiogenesis, tumor invasion, inflammation, and matrix remodeling during wound healing and development has been presented [see Y. Koshelnick et al., “Mechanisms of signaling through Urokinase Receptor and the Cellular Response,” Thrombosis and Haemostasis 82: 305-311 (1999) and F. Blasi, “Proteolysis, Cell Adhesion, Chemotaxis, and Invasiveness Are Regulated by the u-PA-u-PAR-PAI-i System,” Thrombosis and Haemostasis 82: 298-304 (1999)]. Thus, specific antagonists of the binding of u-PA to u-PAR inhibit cell-surface plasminogen activation, tumor growth, and angiogenesis in both in vitro and in vivo models.

H. N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth.

Members of the class of HMG-CoA reductase inhibitors, known as the “statins,” have been shown to inhibit osteoclastic bone resorption in vitro [see J. E. Fisher et al., “Alendronate mechanism of action: geranylgeraniol, an intermediate in the mevalonate pathway, prevents inhibition of osteoclast formation, bone resorption, and kinase activation in vitro,” PNAS USA 96: 133-137 (1999) and S. P. Luckman et al., “Nitrogen-containing bisphosphonates inhibit mevalonate pathway and prevent post-translantional prenylation of GTP-binding proteins, including RAS,” JBMR 13: 581-589 (1998)]. In addition, they have been found to stimulate new bone formation, possibly by induction of BMP-2 [see G. Mundy et al, “Stimulation of bone formation in vitro and in rodents by statins,” Science 286: 1946-1949 (1999) and J. E. Fisher et al, “In vivo effects of bisphosphonates on the osteoclast mevalonate pathway,” Endocrinol 141: 4793-4796 (2000)]. Therefore, the statins hold promise for the treatment of osteoporosis. Nonlimiting examples of statins are lovastatin, simvastatin, atorvastatin, pravastatin, fluvastatin, cerivastatin, and rosuvastatin.

Bone resorption bears similarities to inflammation in which p38 kinase has a rate-limiting function. It has been shown that p38 kinase inhbitors block osteoclast activity and may hold potential as antiresorptive agents in addition to treating arthritis [see A. M. Bader et al., “Pharmacological profile of SB 203580, a selective inhibitor of cytokine suppressive binding protein/p38 kinase, in animal models of arthritis, bone resorption, endotoxin shock and immune function,” J Pharmacol Exp Therap 279: 1453-1461 (1996) and G. A. Rodan and T. J. Martin, “Therapeutic approaches to bone diseases,” Science 289: 1508-1514 (2000).

Activators of the peroxisome proliferator-activated receptor-y γ (PPARγ), such as the thiazolidinediones (TZD's), inhibit osteoclast-like cell formation and bone resorption in vitro. Results reported by R. Okazaki et al. in Endocrinology, 140, pp 5060-5065, (1999) point to a local mechanism on bone marrow cells as well as a systemic one on glucose metabolism. Nonlimiting examples of PPARγ activators include troglitazone, pioglitazone, rosiglitazone, and BRL 49653.

Prenylation inhbitors include, but are not limited to, farnesyl transferase inhibitors, geranylgeranyl transferase inhibitors, and dual farnesyl/greranylgeranyl transferase inhbitors. Geranylgeraniol and its derivatives belong to a class of naturally-occurring compounds known as terpenes. Terpenes are constructed of multiples of five-carbon isoprene units. See Lehninger, A. L., Biochemistry, 1975, pp. 296 and 682-683, which is incorporated by reference herein in its entirety.

For example, geranylgeraniol is a linear terpene containing four isoprene units, corresponding to the following chemical structure.

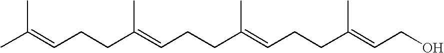

The geranylgeraniol derivative, geranylgeranyl pyrophosphate is an intermediate in the cholesterol biosynthetic pathway and is a substrate in the prenylation of proteins. See J. A. Glomset et 1., Geranylgeranylated proteins, Biochem-Soc-Trans., 1992 May, 20(2): 479-484, which is incorporated by reference herein in its entirety. Certain of these proteins, for example the small GTPases Rac, Rho, and Cdc42, regulate cytoskeletal function.

Ras isoforms are mutated in 20-30% of human cancers making Ras an attractive target for anti-cancer drug development. Since Ras proteins are farnesylated and require prenylation for biological activity, farnesyltransferase inhibitors (FTIs) have been developed to inhibit Ras function and inhibit tumor progression driven by oncogenic Ras, see Law, BK et al., “Farnesyltransferase inhibitor induces rapid growth arrest and blocks p70s6k activation by multiple stimuli,” JBC 2000;275:10796-108010, which is hereby incorporated in its entirety. FTIs have been shown to potently inhibit growth of tumors in mouse models as well as the growth of tumor cells and nontransformed cell lines in culture. FTIs block the growth of tumors with K-ras mutations, other ras mutations as well as those that have no ras mutations, see, Barinaga, M., “From Bench to Bedside,” Science 1997;278:1036-1039, which is hereby incorporated in its entirety. The combination of aminobisphosphonates and FTIs have been shown to synergistically inhibit the prenylation and function of proteins involved in tumor cell invasiveness and mestastasis, see, Andela V., et al., “Synergistic suppression of metastatic phenotype in vitro and metastasis in vivo by a bisphosphonate plus a farnesyl transferase inhibitor,” J Bone Miner Res 2001;16(suppl 1): S1920, which is hereby incorporated in its entirety. The combination of a FTI and a cathepsin K inhibitor is possible in the treatment and prevention of bone metastases as cathepsin K is expressed both by breast cancers, see, Cleaton-Roberts M, et al., “Chracterization of polyclonal and monoclonal anti-cathepsin K antibodies and the demonstration of the expression of this protein in primary breast carcinoma and skeletal metastasis,” J Bone Miner Res 1999; 14(suppl 1):S358, which is hereby incorporated in its entirety, and prostate cancer, see, Brubaker KD, et al., “Expression and activity of cathepsin K in prostate cancer,” J Bone Miner Res 2001;16(suppl 1):S334, which is hereby incorporated in its entirety.

Prostaglandin inhibitors are routinely used in the treatment of arthritis. These agents include NSAIDs (dual COX-1/COX-2) and selective COX-2 inhibitors. While these agents may have some effect on bone metabolism (NH Bell, et al., “Diclofenac inhibits bone resorption in postmenopausal women,” Am J Med 96: 349-353 (1994); K. Igarashi et al., “The cyclooxygenase-2 inhibitor, celecoxib, inhibits bone resorption elicited by interleukin-1-β, endothelin-1 and triiodothyronine, but not by prostaglandin E₂ in neonatal mouse calvarial organ cultures,” JBMR 15 (suppl 1), S272 (2000)), they may also be combined with antiresorptives to treat bone loss associated with arthritis.

Antagonists of the Ca²⁺ receptor, a calcilytic, would mimic a state of hypocalcemia and thereby stimulate PTH secretion. If repeated on a daily basis this may have anabolic effects on bone. Results reported by M. Gowen et al. in JCI, 105, pp 1595-1604, (2000) demonstrate that antagonizing the parathyroid calcium receptor stimulates parathyroid hormone secretion and bone formation in osteopenic rats. Nonlimiting examples of calcilytics include NPS 2143.

Members of the TGF-β superfamily, including bone morphogenetic proteins (BMPs) and their receptors play an important role in the formation of skeletal tissues with novel clinical applications including local application for fracture healing and repair of bone defects [see T. Sakou, “Bone morphogenetic proteins: from basic studies to clinical applications,” Bone 22: 591-603 (1998)]. They may also serve to treat bone diseases, especially severe osteoporosis. Given the peptide nature of growth factors, which limits their oral administration, gene therapy may be required for delivery. This may be accomplished by transfecting osteoblasts with a vector containing the BMP gene [see J. Bonadio et al., “Localized, direct plasmid gene delivery in vivo: prolonged therapy results in reproducible tissue regeneration,” Nature Medicine 5: 753-759 (1999)].

The intermittent administration of parathyroid hormone (PTH) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by RM Neer et al., in New Eng J Med 344 1434-1441 (2001).

Parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

Prostaglandin E (PGE) blocks osteoblast apoptosis [see M. Machwate et al., “Sphingosine kinase mediates cyclic AMP suppression of apoptosis in rat periosteal cells” Mol Pharmacol 54: 70-77]; therefore, prostanoid or non-prostanoid analogues of PGE, or agents that work through this pathway may have anabolic effects on bone (see HZ Ke et al, “Discovery of a non-prostanoid EP2 receptor selective prostaglandin E2 (PGE2) agonist that mimics the local anabolic effects of PGE2,” JBMR 15 (suppl 1) S377 (2000)).

Inducers of osteoblast Cbfa1 may be instrumental in treating osteoporosis. Cbfa1 is a critical regulator of both osteoblast differentiation and function [see G. Karsenty, “Role of Cbfa1 in osteoblast differentiation and function,” Cell Dev Biol 11: 343-346 (2000)]. Furthermore, transgenic mice that overexpress Cbfa1 have higher bone mass compared to age-matched littermates and this phenotype increases with time [see M. H. Priemel et al., “Increased bone formation in Cbfa1 overexpressing mice,” JBMR 14 (suppl 1) S171 (1999)].

Pharmacological antagonism of Dkk action at LRP5 in the osteoblast may increase bone density and offer promise in treating osteoporosis/osteopenia [MS Patel, G Karsenty, “Regulation of bone formation and vision by LRP5” NEJM 346: 1572-1574 (2002)]. Lipoprotein receptor-related protein 5 (LRP5) functions as a Wnt co-receptor in the osteoblast signaling pathway, and gain of function mutations in LRP5 increase Wnt signaling by impairing the action of normal antagonists of the Wnt pathway, thus resulting in a high bone mass phenotype [see L. M. Boyden et al., “High bone mass due to mutation in LDL-receptor-related protein 5,” NEJM 346: 1513-1521 (2002)]. The Wnt signaling pathway can be antagonized by the secreted Dickkopf (Dkk) family of proteins suggesting that Dkk antagonists, either at the prereceptor level or through the inhibition of binding or action of LRP5, may hold promise in treating/preventing osteoporosis.

Antagonists of hypothalamic Y2 receptors may be useful in treating osteoporosis. Hypothalamic Y2 receptors are involved in the tonic inhibition of bone formation with Y2 receptor-deficient mice having a twofold increase in trabecular bone volume as well as greater trabecular number and thickness compared to control mice [PA Baldock et al., “Hypothalamic Y2 receptors regulate bone formation”, JCI 109: 915-921 (2002)].

The pharmacological manipulation of the leptin pathway may yield novel therapeutics to prevent and treat osteoporosis [P Ducy, et al., “Leptin inhibits bone formation through a hypothalamic relay: a central control of bone mass”, Cell 100: 197-207 (2000)]. The absence of leptin signaling is associated with a high bone mass phenotype in both the ob/ob and db/db mice due to an increase in bone matrix deposition and, further, highlights the central nature of bone mass control.

Antagonists that block the sclerostin/BMP interaction hold promise as anabolic agents for treating osteoporosis [DG Winkler et al., “Sclerostin, the protein product of the Sclerostosis gene (SOST) and a key regulator of bone matrix formation, binds to BMPs and antagonizes their function”, JBMR 16 (Suppl 1) S322 (2002)]. Sclerostosis, a skeletal dysplasia characterized by increased bone formation, results from loss of the SOST gene product, sclerostin. Sclerostin has been demonstrated to block BMP-6-induced ALP activity in a dose-dependent manner, therefore sclerostin antagonists may increase bone formation through unopposed anabolic BMP activity [RL van Bezooijen et al., “BMP-antagonist sclerostin is expressed in mineralized bone and blocks BMP-induced bone formation in vitro”, JBMR 16 (Suppl 1) S163 (2002)].

Agonists of the P2X7 receptor offer therapeutic potential in treating and preventing osteoporosis [HZ Ke et al., “P2X7 receptor regulates bone formation and bone resorption”, JBMR 17 (Suppl 1) S150 (2002)]. The P2X7R is an ATP-gated ion channel expressed in osteoclasts and a subpopulation of osteoblasts, and is believed to play a stimulating role in periosteal and cancellous bone formation as well as an inhibiting role in cancellous bone resorption.

Inhibitors of the CIC-7 chloride channel may potentially be used to treat and prevent osteoporosis. Mice deficient in the ubiquitously expressed CIC-7 chloride channel develop severe osteopetrosis due to the inability to resorb bone [U Kornak et al., “Loss of the CIC-7 chloride channel leads to osteopetrosis in mice and men”, Cell 104: 205-215 (2001)}. Mutations in this CIC-7 gene have also been described in humans with osteopetrosis [SG Waguespack et al., “Characterization of mutations in the chloride channel 7 (CICN7) gene that cause autosomal dominant osteopetrosis, Type II”, JBMR 17 (Suppl 1) S144 (2002)].

Tumor Necrosis Factor alpha (“TNFa”) plays a role in the pathogenesis of postemenopausal osteoporosis through the activation of osteoclastic bone resorption. It has been shown that women with postmenopausal osteoporosis have higher serum levels of TNFa than premenopausal women, and that this correlates with age and menopausal age, see, Uzun H, et al., “The study of interleukin-1b, interleukin-6, tumor necrosis factor-a role in postmenopausal osteoporosis.,” J Bone Miner Res 1999; 14 (suppl 1):S520, which is hereby incorporated in its entirety. TNF-a and IL-1 can be detected in the synovial fluid of patients with rheumatoid arthritis and are likely to have a primary role in the pathogenesis of rheumatoid arthritis, see, “Choy EHS, Panayi GS. Cytokine pathways and joint inflammation in rheumatoid arthritis,” N Eng J Med 2001;344:907-916, which is hereby incorporated in its entirety. TNFa inhibitors, etanercept and infliximab are the first biological-response modifiers approved for the treatment of rheumatoid arthritis, see, DM Lee, ME, “Weinblatt Rheumatoid arthritis,” Lancet 2001;358:903-911, which is hereby incorporated in its entirety. Furthermore, cathepsin K is present in osteoclast-like cells and may play a role in erosions in subchondral bone and cartilage, as well as at the pannus-cartilage junction [see M. Kaneko et al., Expression of proteinases and inflammatory cytokines in subchondral bone regions in the destructive joint of rheumatoid arthritis. Rheumatology 2001;40:247-255]. Hence, the combination of TNF blockers and cathepsin K inhibitors may treat rheumatoid arthritis and associated underlying bone loss.

The present invention is also directed to combinations of the compounds of the present invention with one or more agents useful in the prevention or treatment of osteoporosis, arthritic conditions, osteoarthritis, rheumatoid arthritis, tumor metastases, breast cancer, prostate cancer, metastatic bone disease, Paget's disease, and other metabolic bone disorders characterized by increased bone resorption. For example, the compounds of the instant invention may be effectively administered in combination with effective amounts of other agents such as an organic bisphosphonate, an estrogen receptor modulator, an androgen receptor modulator, an αᵥβ₃ integrin receptor antagonist, an HMG-CoA reductase inhibitor, a PPARγ activator, a VEGF receptor antagonist, or an inhibitor of the osteoclast proton ATPase.

Additional illustrations of the invention are methods of treating cancer or metastatic tumor growth in a mammal in need thereof, comprising administering to the mammal a therapeutically effective amount of a compound described above and one or more agents known to be cytotoxic/antiproliferative. Also, the compounds of the present invention can be administered in combination with radiation therapy for treating cancer and metastatic tumor growth.

In addition, the cathepsin K inhibitor of the present invention may be effectively administered in combination with a growth hormone secretagogue in the therapeutic or prophylactic treatment of disorders in calcium or phosphate metabolism and associated diseases. These diseases include conditions which can benefit from a reduction in bone resorption. A reduction in bone resorption should improve the balance between resorption and formation, reduce bone loss or result in bone augmentation. A reduction in bone resorption can alleviate the pain associated with osteolytic lesions and reduce the incidence and/or growth of those lesions. These diseases include: osteoporosis (including estrogen deficiency, immobilization, glucocorticoid-induced and senile), osteodystrophy, Paget's disease, myositis ossificans, Bechterew's disease, malignant hypercalcemia, metastatic bone disease, periodontal disease, cholelithiasis, nephrolithiasis, urolithiasis, urinary calculus, hardening of the arteries (sclerosis), arthritis, bursitis, and neuritis. Increased bone resorption can be accompanied by pathologically high calcium and phosphate concentrations in the plasma, which would be alleviated by this treatment. Similarly, the present invention would be useful in increasing bone mass in patients with growth hormone deficiency. Thus, preferred combinations are simultaneous or alternating treatments of a cathepsin K inhibitor of the present invention and a growth hormone secretagogue, optionally including a third component comprising an organic bisphosphonate, preferably alendronate monosodium trihydrate.

In accordance with the method of the present invention, the individual components of the combination can be administered separately at different times during the course of therapy or concurrently in divided or single combination forms. The instant invention is therefore to be understood as embracing all such regimes of simultaneous or alternating treatment, and the term “administering” is to be interpreted accordingly. It will be understood that the scope of combinations of the compounds of this invention with other agents useful for treating any conditions noted above includes in principle any combination with any pharmaceutical composition useful for treating osteoporosis.

As used herein, the term “composition” is intended to encompass a product comprising the specified ingredients in the specified amounts, as well as any product which results, directly or indirectly, from combination of the specified ingredients in the specified amounts.

As used herein, the term “pharmaceutically acceptable salts” includes the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

The compositions of the present invention can be administered in such oral dosage forms as tablets, capsules (each of which includes sustained release or timed release formulations), pills, powders, granules, elixirs, tinctures, sterile solutions or suspensions, syrups and emulsions. Likewise, it may also be administered in intravenous (bolus or infusion), intraperitoneal, topical (e.g., ocular eyedrop), intranasal, inhaled, subcutaneous, intramuscular or transdermal (e.g., patch) form, metered aerosol or liquid sprays, drops, ampoules, auto-injector devices or suppositories all using forms well known to those of ordinary skill in the pharmaceutical arts. An effective but non-toxic amount of the compositions desired can be employed. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen utilizing the compositions of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Advantageously, the compound of the present invention may be administered in a single quarterly, monthly, weekly or daily dose, or the total daily dosage may be administered in divided doses of two, three or four times daily. Furthermore, the compound of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

Oral dosages of the cathepsin K inhibitors of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion.

Oral dosages of the active ingredients of the present invention which can be combined with the cathepsin K inhibitors of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion.

The precise dosage of the bisphosphonate will vary with the dosing schedule, the oral potency of the particular bisphosphonate chosen, the age, size, sex and condition of the mammal or human, the nature and severity of the disorder to be treated, and other relevant medical and physical factors. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

Oral dosages of the estrogen receptor modulators of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion.

Generally, the daily dosage of the androgen receptor modulators of the present invention may be varied over the range from 0.01 to 1000 mg per adult human per day. Most preferably, dosages range from 0.1 to 200 mg/day. For oral administration, the compositions are preferably provided in the form of tablets containing 0.01 to 1000 mg, particularly 0.01, 0.05, 0.1, 0.5, 1.0, 2.5, 3.0, 5.0, 6.0, 10.0, 15.0, 25.0, 50.0, 75, 100, 125, 150, 175, 180, 200, 225, and 500 milligrams of the active ingredient for the symptomatic adjustment of the dosage to the patient to be treated.

Oral dosages of the ATPase inhibitors of the present invention, when used for the indicated effects, will range between about 0.01 mg per kg of body weight per day (mg/kg/day) to about 100 mg/kg/day, preferably 0.01 to 10 mg/kg/day, and most preferably 1.0 to 10.0 mg/kg/day. For oral administration, the compositions are preferably provided in the form of tablets containing 5.0 and 10.0 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion.

In particular, for daily dosing, the amounts of the HMG-CoA reductase inhibitor can be the same or similar to those amounts which are employed for anti-hypercholesterolemic treatment and which are described in the Physicians' Desk Reference (PDR), 52^{nd} Ed. of the PDR, 1998 (Medical Economics Co), which is incorporated by reference herein in its entirety. For the additional active agents, the doses can be the same or similar to those amounts which are known in the art.

The HMG-CoA reductase inhibitors can be administered via a wide variety of routes including oral administration, intravenous administration, intranasal administration, injections, ocular administration, and the like. A preferred route of delivery is oral administration.

Oral dosage amounts of the HMG-CoA reductase inhibitor are from about 1 to 200 mg/day, and more preferably from about 5 to 160 mg/day. However, dosage amounts will vary depending on the potency of the specific HMG-CoA reductase inhibitor used as well as other factors as noted above. An HMG-CoA reductase inhibitor which has sufficiently greater potency may be given in sub-milligram daily dosages. The HMG-CoA reductase inhibitor may be administered from 1 to 4 times per day, and preferably once per day.

For example, the daily dosage amount for simvastatin can be selected from 5 mg, 10 mg, 20 mg, 40 mg, and 80 mg; for lovastatin, 10 mg, 20 mg, 40 mg and 80 mg; for fluvastatin sodium, 20 mg, 40 mg and 80 mg; for pravastatin sodium, 10 mg, 20 mg, and 40 mg; and for atorvastatin calcium, 10 mg, 20 mg, and 40 mg.

Oral dosages of the αvβ3 inhibitors of present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 10 mg to about 400 mg of active ingredient. Intravenously, the most preferred doses will range from about 0.1 to about 10 mg/kg/minute during a constant rate infusion.

Oral dosages of PTH, when used for the indicated effects, will range between about 0.001 μg per kg of body weight per day (mg/kg/day) to about 10 mg/kg/day, preferably 0.001 to 10 mg/kg/day. For subcutaneous administration, the PTH is provided in doses of 20 μg and 40 μg of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion.

Methods of the Present Invention

The present invention comprises methods for treating abnormal bone resorption in mammals. The present invention also comprises methods for preventing abnormal bone resorption in mammals. In preferred embodiments of the present invention, the mammal is a human.

The methods and compositions of the present invention are useful for both treating and preventing abnormal bone resorption and conditions associated therewith. Conditions associated with abnormal bone resorption include both generalized and localized bone loss. Also, the creation of bone having an abnormal structure, as in Paget's disease, can be associated with abnormal bone resorption. The term “generalized bone loss” means bone loss at multiple skeletal sites or throughout the skeletal system. The term “localized bone loss” means bone loss at one or more specific, defined skeletal sites.

Generalized boss loss is often associated with osteoporosis; Osteoporosis is most common in post-menopausal women, wherein estrogen production has been greatly diminished. However, osteoporosis can also be steroid-induced and has been observed in males due to age. Osteoporosis can be induced by disease, e.g. rheumatoid arthritis, it can be induced by secondary causes, e.g., glucocorticoid therapy, or it can come about with no identifiable cause, i.e. idiopathic osteoporosis. In the present invention, preferred methods include the treatment or prevention of abnormal bone resorption in osteoporotic humans.

Localized bone loss has been associated with periodontal disease, with bone fractures, and with periprosthetic osteolysis (in other words where bone resorption has occurred in proximity to a prosthetic implant).

Generalized or localized bone loss can occur from disuse, which is often a problem for those confined to a bed or a wheelchair, or for those who have an immobilized limb set in a cast or in traction.

The methods and compositions of the present invention are useful for treating or preventing the following conditions or disease states: osteoporosis, which can include post-menopausal osteoporosis, corticosteroid-induced osteoporosis, male osteoporosis, disease-induced osteoporosis, idiopathic osteoporosis; Paget's disease; abnormally increased bone turnover; osteomalacia; periodontal disease; localized bone loss associated with periprosthetic osteolysis; and bone fractures.

The methods and compositions of the present invention are useful for inhibiting bone resorption. In addition, the methods and compositions of the present invention are useful for increasing bone mineral density in a patient. Also, the methods and compositions of the present invention are useful for reducing the risk of vertebral or nonvertebral fractures.

The methods and compositions of the present invention are useful for reducing bone turnover resorption markers. Bone turnover may be assessed by measurement of various serum or urinary markers, such as urinary hydroxyproline (Hyp), Urinary total pyrdinoline, Urinary total deoxypyridinoline (dPyr), Urinary free pyridinoline (f-Pyr), Urinary free deoxypyridinoline (f-dPyr), Urinary collagen type 1 cross-linked N-telopeptide (NTx), Urinary collagen type 1 cross-linked C-telopeptide (CTx), Serum carboxyterminal telopeptide of type 1 collagen (1TCP). Measurement of one or more of these markers can reflect a patient's fracture risk, and can be used in conjunction with bone mineral density testing to screen or diagnose bone disease; high levels of bone turnover can be indicative of a disease state in bone. In addition to initial diagnosis, treatment response can be assessed via measurement of bone turnover markers. See, for example, Watts, N. B. “Clinical utility of biochemical markers of bone remodeling,” Clin Chem. 1999; Miller, P. D., Baran, D. T. Bilezekian J., et al., “Practical clinical application of biochemical markers of bone turnover,” J Clin Densiometry 1999.

The compositions and methods of the present invention are useful for treating or preventing arthritic conditions, especially for treating or preventing osteoarthritis and rheumatoid arthritis, including the prevention of subchondral bone resorption, osteophyte formation and ultimately joint deterioration/destruction, and in the prevention and treatment of metastatic bone disease.

“Arthritic condition” or “arthritic conditions” refers to a disease wherein inflammatory lesions are confined to the joints or any inflammatory conditions of the joints, most notably osteoarthritis and rheumatoid arthritis (Academic Press Dictionary of Science Technology; Academic Press; 1st edition, Jan. 15, 1992). The compositions of the present invention are also useful, alone or in combination, to treat or prevent arthritic conditions, such as Behcet's disease; bursitis and tendinitis; CPPD deposition disease; carpal tunnel syndrome; Ehlers-Danlos syndrome; fibromyalgia; gout; infectious arthritis; inflammatory bowel disease; juvenile arthritis; lupus erythematosus; lyme disease; marfan syndrome; myositis; osteoarthritis; osteogenesis imperfecta; osteonecrosis; polyarteritis; polymyalgia rheumatica; psoriatic arthritis; Raynaud's phenomenon; reflex sympathetic dystrophy syndrome; Reiter's syndrome: rheumatoid arthritis: scleroderma; and Sjogren's syndrome. An embodiment of the invention encompasses the treatment or prevention of an arthrtic condition which comprises administering a therapeutically effective amount of a composition of the present invention. A subembodiment is the treatment or prevention of osteoarthritis which comprises administering a therapeutically effective amount of a composition of the present invention. See: Cutolo M, Seriolo B, Villaggio B, Pizzorni C, Craviotto C, Sulli A. Ann. N. Y. Acad. Sci. 2002 June;966:131-42; Cutolo, M. Rheum Dis Clin North Am 2000 November;26(4):881-95; Bijisma JW, Van den Brink HR. Am J Reprod Immunol 1992 Oct-Dec;28(3-4):231-4; Jansson L, Holmdahl R.; Arthritis Rheum 2001 September;44(9):2168-75; and Purdie DW. Br Med Bull 2000;56(3):809-23. Also, see Merck Manual, 17th edition, pp. 449-451.

When used in combination to treat arthritic conditions, the compositions of the present invention can be used with any of the drugs diclosed herein as useful for combination therapy, or can be used with drugs known to treat or prevent arthritic conditions, such as corticosteroids, cytoxic drugs (or other disease modifying or remission inducing drugs), gold treatment, methotrexate, NSAIDs, and COX-2 inhibitors.

Osteoarthritis (OA) is a connective tissue disease, with pathology arising from mechanical insult-induced articular cartilage degeneration, a limited synovial inflammatory response, and subchondral bone remodeling. The net outcome of these activities is joint deformity secondary to erosion of the articular surface, peri-articular endochondral ossification/osteophytosis, and subchondral bony sclerosis and cyst formation [R Oettmeier, K. Abendroth “Osteoarthritis and bone: osteologic types of osteoarthritis of the hip”, Skeletal Radiol 18: 165-174 (1989)]. Although, OA is considered primarily a cartilaginous disorder, it is accompanied by well-defined changes in trabecular bone of the joint, and it is possible that the subchondral bone changes may potentially contribute to the initiation and progression of OA [K Senior. “Osteoarthritis research: on the verge of a revolution? Lancet 355: 208 (2000)].

Cathepsin K is highly expressed in giant cells that form within OA synovial tissue and may principally digest both bone and cartilage fragments sheered from joint surfaces in OA [RA Dodds et al., “Expression of cathepsin K messenger RNA in giant cells and their precursors in human osteoarthritic synovial tissues”, Arth Rheum 42: 1588-1593 (1999)]. In addition, cathepsin K is known to be induced in phenotypically altered chondrocytes in OA and is potentially instrumental in degrading the superficial gliding surfaces of articular hyaline cartilage in OA [YT Konttinen et al., “Acidic cysteine endoproteinase cathepsin K in the degeneration of the superficial articular hyaline cartilage in osteoarthritis”, Arth Rheum 46: 953-960 (2002)]. Cathepsin K has also been demonstrated to cleave type II collagen [W Kafieneh et al., Human cathepsin K cleaves native type-I and type-II collagens at the n-terminal end of the triple-helix”, Biochem J 331: 727-732 (1998)] which is almost exclusively localized to cartilage. Therefore, cathepsin K inhibitors may have potential utility as chondroprotective agents in the prevention and treatment of osteoarthritis [YT Konttinen et al., “Acidic cysteine endoproteinase cathepsin K in the degeneration of the superficial articular hyaline cartilage in osteoarthritis”, Arth Rheum 46: 953-960 (2002)].

Rheumatoid arthritis (RA) is a disease of joints characterized by chronic inflammation of the synovium [ED Harris, Jr. “Rheumatoid arthritis: pathophysiology and implications for therapy”, NEJM 322: 1277-1289 (1990)] with formation of an inflammatory cell proliferation termed the pannus, which contains many growth factors and inflammatory cytokines, including IL-1β, TNF-α and matrix-degrading proteinases. Cathepsin K expression is stimulated by both IL-10 and TNF-α in human RA synovial fibroblasts implicating a potential role of this protease in the pathogenesis of RA [W—S Hou et al., “Comparison of cathepsins K and S expression within the rheumatoid and osteoarthritic synovium” Arth Rheum 46: 663-674 (2002)]. Cathepsin K may thus represent a therapeutic target for the treatment of cartilage degradation in RA, either alone or combination with other agents.

Metastatic bone disease (MBD) is a common complication of breast, prostate and other cancers that arises when tumor cells optimize the microenvironment to favor osteoclast-mediated bone resorption. Cathepsin K inhibitors may, therefore, may be used to treat tumor osteolysis by targeting the osteoclast. Cathepsin K is also expressed on both prostate and breast cancer cells and could be involved in the establishment of bone metastases by initiating and enhancing collagen degradation [KD Brubaker et al., “Expression and activity of cathepsin K in prostate cancer” JBMR 16 (Suppl 1) S334 (2001)]. Therefore, in addition to treating and prevening MBD either alone or in combination with other agents, cathepsin K inhibitors may hold promise in preventing tumor cell attachment to bone or other tissues.

The compositions and methods of the present invention are administered and carried out until the desired therapeutic effect is achieved.

What is claimed is:

1. A pharmaceutical composition comprising a cathpesin K inhibitor and one or more active ingredients selected from the following:
a) an organic bisphosphonate or a pharmaceutically acceptable salt or ester thereof,
b) an estrogen receptor modulator,
c) an androgen receptor modulator,
d) a steroid with mixed estrogenic-progestogenic-androgenic properties,
e) a cytotoxic antiproliferative agent,
f) a matrix metalloproteinase inhibitor,
g) an inhibitor of epidermal-derived growth factors,
h) an inhibitor of fibroblast-derived growth factors,
i) an inhibitor of platelet-derived growth factors,
j) an inhibitor of VEGF,
k) an antibody to a growth factor, an antibody to a growth factor receptor, I) an inhibitor of Flk-1/KDR,
m) an inhibitor of Flt-1,
n) an inhibitor of Tck/Tie-2,
o) an inhibitor of Tie-1,
p) αvβ3 receptor antagonist,
q) growth hormone,
r) a growth hormone analogue,
s) a growth hormone secretagogue,
t) an inhibitor of osteoclast ATPase,
u) an inhibitor of urokinase plasminogen activator,
v) a tumor-specific antibody-interleukin-2 fusion protein,
w) an inhibitor of HMG-CoA reductase,
x) an inhibitor of p38 kinase,
y) an activator of the peroxisome proliferator-activated receptor-y,
z) a prenylation inhibitor,
aa) a COX-1 inhibitor,
bb) COX-2 inhibitor,
cc) a dual COX-1/COX-2 inhibitor,
dd) a calcilytic,
ee) growth factors,
ff) Parathyroid hormone (PTH),
gg) PTH fragments,
hh) PTH analogues,
ii) Parathyroid hormone-related protein (PTHrP),
jj) PTHrP fragments,
kk) PTHrP analogues,
ll) a prostanoid EP2 receptor agonist,
mm) a non-prostanoid EP2 receptor agonist,
nn) inducers of osteoblastic Cbfa-1,
oo) a bone anabolic agent that directly stimulates osteoblastic activity,
pp) an inhibitor of tumor necrosis factor-α, and
qq) an anti-inflammatory agent,
rr) a Dkk inhibitor or a stimulator of the Wnt signaling pathway,
ss) a Y2 receptor antagonist,
tt) a central inhibitor of leptin signaling,
uu) a sclerostin antagonist,
vv) a P2X7 receptor agonist,
ww) a CIC-7 inhibitor,
and the pharmaceutically acceptable salts and mixtures thereof.

2. The pharmaceutical composition of claim 1 comprising a cathepsin K inhibitor and one more active ingredients selected from the following:
a) an organic bisphosphonate or a pharmaceutically acceptable salt or ester thereof,
b) an estrogen receptor modulator,
c) an androgen receptor modulator,
d) an inhibitor of osteoclast proton ATPase,
e) an inhibitor of HMG-CoA reductase,
f) an αᵥβ₃ receptor antagonist,
g) an osteoblast anabolic agent,
and the pharmaceutically acceptable salts and mixtures thereof.

3. The pharmaceutical composition of claim 2 wherein the organic bisphosphonate is selected from the group consisting of include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof.

4. The pharmaceutical composition of claim 3 wherein the organic bisphosphonate is alendronate.

5. The pharmaceutical composition of claim 2 wherein the estrogen receptor modulator is selected from the group consisting of estrogen, progestogen, estradiol, droloxifene, raloxifene, lasofoxifene, TSE-424, tamoxifen and the pharmaceutically acceptable salts thereof.

6. The pharmaceutical composition of claim 2 wherein the osteoblast anabolic agent is selected from the group consisting of PTH, PTH fragments, PTH analogues, BMP and the pharmaceutically acceptable salts thereof.

7. The pharmaceutical composition of claim 2 wherein the HMG-CoA reductase inhibitor is selected from the group consisting of lovastatin, simvastatin, atorvastatin, pravastatin, fluvastatin, cerivastatin, rosuvastatin and the pharmaceutically acceptable salts thereof.

8. The pharmaceutical composition of claim 7 wherein the HMG-CoA reductase inhibitor is selected from the group consisting of lovastatin, simvastatin, and the pharmaceutically acceptable salts thereof.

9. The pharmaceutical composition which is prepared by combining a cathepsin K inhibitor and one more active ingredients selected from the following:
a) an organic bisphosphonate or a pharmaceutically acceptable salt or ester thereof,
b) an estrogen receptor modulator,
c) an androgen receptor modulator,
d) an inhibitor of osteoclast proton ATPase,
e) an inhibitor of HMG-CoA reductase,
f) an αᵥβ₃ receptor antagonist,
g) an osteoblast anabolic agent,
and the pharmaceutically acceptable salts and mixtures thereof.

10. A method for treating or preventing osteoporosis in a mammal in need thereof comprising administering a pharmaceutical composition comprising a cathepsin K inhibitor and one more active ingredients selected from the following:
a) an organic bisphosphonate or a pharmaceutically acceptable salt or ester thereof,
b) an estrogen receptor modulator,
c) an androgen receptor modulator,
d) a steroid with mixed estrogenic-progestogenic-androgenic properties,
e) a cytotoxic antiproliferative agent,
f) a matrix metalloproteinase inhibitor,
g) an inhibitor of epidermal-derived growth factors,
h) an inhibitor of fibroblast-derived growth factors,
i) an inhibitor of platelet-derived growth factors,
j) an inhibitor of VEGF,
k) an antibody to a growth factor, an antibody to a growth factor receptor,
l) an inhibitor of Flk-1/KDR,
m) an inhibitor of Flt-1,
n) an inhibitor of Tck/Tie-2,
o) an inhibitor of Tie-1,
p) αvβ3 receptor antagonist,
q) growth hormone,
r) a growth hormone analogue,
s) a growth hormone secretagogue,
t) an inhibitor of osteoclast ATPase,
u) an inhibitor of urokinase plasminogen activator,
v) a tumor-specific antibody-interleukin-2 fusion protein,
w) an inhibitor of HMG-CoA reductase,
x) an inhibitor of p38 kinase,
y) an activator of the peroxisome proliferator-activated receptor-y,
z) a prenylation inhibitor,
aa) a COX-1 inhibitor,
bb) COX-2 inhibitor,
cc) a dual COX-1/COX-2 inhibitor,
dd) a calcilytic,
ee) growth factors,
ff) Parathyroid hormone (PTH),
gg) PTH fragments,
hh) PTH analogues,
ii) Parathyroid hormone-related protein (PTHrP),
jj) PTHrP fragments,
kk) PTHrP analogues,
ll) a prostanoid EP2 receptor agonist,
mm) a non-prostanoid EP2 receptor agonist,
nn) inducers of osteoblastic Cbfa-1,
oo) a bone anabolic agent that directly stimulates osteoblastic activity,
pp) an inhibitor of tumor necrosis factor-a, and
qq) an anti-inflammatory agent,
rr) a Dkk inhibitor or a stimulator of the Wnt signaling pathway,
ss) a Y2 receptor antagonist,
tt) a central inhibitor of leptin signaling,
uu) a sclerostin antagonist,
vv) a P2X7 receptor agonist,
ww) a CIC-7 inhibitor,
and the pharmaceutically acceptable salts and mixtures thereof.

11. The method of claim 10 comprising a cathepsin K inhibitor and one more active ingredients selected from the following:
a) an organic bisphosphonate or a pharmaceutically acceptable salt or ester thereof,
b) an estrogen receptor modulator,
c) an androgen receptor modulator,
d) an inhibitor of osteoclast proton ATPase,
e) an inhibitor of HMG-CoA reductase,
f) an αᵥβ₃ receptor antagonist,
g) an osteoblast anabolic agent;
and the pharmaceutically acceptable salts and mixtures thereof.

12. A method of treating or preventing an arthritic condition in a mammal in need thereof comprising administering a pharmaceutical composition comprising a cathepsin K inhibitor and one more active ingredients selected from the following:
a) an organic bisphosphonate or a pharmaceutically acceptable salt or ester thereof,
b) an estrogen receptor modulator,
c) an androgen receptor modulator,
d) a steroid with mixed estrogenic-progestogenic-androgenic properties,
e) a cytotoxic antiproliferative agent,
f) a matrix metalloproteinase inhibitor,
g) an inhibitor of epidermal-derived growth factors,
h) an inhibitor of fibroblast-derived growth factors,
i) an inhibitor of platelet-derived growth factors,
j) an inhibitor of VEGF,
k) an antibody to a growth factor, an antibody to a growth factor receptor,
l) an inhibitor of Flk-1/KDR,
m) an inhibitor of Flt-1,
n) an inhibitor of Tck/Tie-2,
o) an inhibitor of Tie-1,
p) αvβ3 receptor antagonist,
q) growth hormone,
r) a growth hormone analogue,
s) a growth hormone secretagogue,
t) an inhibitor of osteoclast ATPase,
u) an inhibitor of urokinase plasminogen activator,
v) a tumor-specific antibody-interleukin-2 fusion protein,
w) an inhibitor of HMG-CoA reductase,
x) an inhibitor of p38 kinase,
y) an activator of the peroxisome proliferator-activated receptor-y,
z) a prenylation inhibitor,
aa) a COX-1 inhibitor,
bb) COX-2 inhibitor,
cc) a dual COX-1/COX-2 inhibitor,
dd) a calcilytic,
ee) growth factors,
ff) Parathyroid hormone (PTH),
gg) PTH fragments,
hh) PTH analogues,
ii) Parathyroid hormone-related protein (PTHrP),
jj) PTHrP fragments,
kk) PTHrP analogues,
ll) a prostanoid EP2 receptor agonist,
mm) a non-prostanoid EP2 receptor agonist,
nn) inducers of osteoblastic Cbfa-1,
oo) a bone anabolic agent that directly stimulates osteoblastic activity,
pp) an inhibitor of tumor necrosis factor-α, and
qq) an anti-inflammatory agent,
rr) a Dkk inhibitor or a stimulator of the Wnt signaling pathway,
ss) a Y2 receptor antagonist,
tt) a central inhibitor of leptin signaling,
uu) a sclerostin antagonist,
vv) a P2X7 receptor agonist,
ww) a CIC-7 inhibitor,
and the pharmaceutically acceptable salts and mixtures thereof.

13. The method of claim 12 wherein the arthritic condition is osteoarthritis.

14. The method of claim 12 comprising a cathepsin K inhibitor and one more active ingredients selected from the following:
a) an organic bisphosphonate or a pharmaceutically acceptable salt or ester thereof,
b) an estrogen receptor modulator,
c) an androgen receptor modulator,
d) an inhibitor of osteoclast proton ATPase,
e) an inhibitor of HMG-CoA reductase,
f) an αᵥβ₃ receptor antagonist,
g) an osteoblast anabolic agent,
and the pharmaceutically acceptable salts and mixtures thereof.

15. The method of claim 14 wherein the arthritic condition is osteoarthritis.

16. A method of inhibiting bone resorption in a patient by administering a pharmaceutical composition of claim 1.

17. A method of increasing Bone Mineral Density in a patient by administering a pharmaceutical composition of claim 1.

18. A method of reducing the risk of vertebral or nonvertebral fractures in a patient by administering a pharmaceutical composition of claim 1.

19. A method of reducing urinary NTx in a patient by administering a pharmaceutical composition of claim 1.
